(19) United States
(12) Patent Application Publication
(10) Pub. No.: US 2026/0098850 A1
(43) Pub. Date: Apr. 9, 2026

(54) DETECTING MATERIAL CHARACTERISTICS DURING HYDRAULIC OPERATIONS

(71) Applicant: Halliburton Energy Services, Inc., Houston, TX (US)

(72) Inventors: Cameron Michael Kramer, Duncan, OK (US); Carlos Alfredo Vallejo Gordon, Houston, TX (US); Jeremy Rioux, Duncan, OK (US); Alex Brandon Cline, Duncan, OK (US); Shahab Jamali Ghare Tape, Houston, TX (US); Andrew Silas Clyburn, Duncan, OK (US)

(21) Appl. No.: 18/907,030

(22) Filed: Oct. 4, 2024

(51) Int. Cl. G01N 33/28; E21B 43/26

(52) U.S. Cl. CPC G01N 33/2823; E21B 43/2607

(57) ABSTRACT

A method to control hydraulic fracturing operations comprises transporting material to a blender, via a proppant transportation system, during the hydraulic fracturing operations, wherein the material includes proppant. The method comprises obtaining, via one or more sensors, media content of the material as the material is transported to the blender. The method comprises determining, via a learning machine, one or more material characteristics based on the media content.

### FIELD

Some implementations relate generally to the field of hydraulic fracturing operations and more particularly to the field of determining characteristics of proppant utilized in hydraulic fracturing operations.

### BACKGROUND

In hydrocarbon recovery operations, fluid and proppant may be pumped into a wellbore to hydraulically fracture a subsurface formation. A fracturing spread may include fracturing pumps configured to pump fluid and proppant into the wellbore, a blender (to mix proppant, fluid, chemical, etc.), and a proppant transportation system to transport proppant to the blender. The pump rate and pressure from the fluid may fracture the subsurface formation, creating a conduit for the fluid in the subsurface formation to flow to the wellbore and ultimately to the surface. Proppant, such as natural sand, man-made sand, or other man-made materials, may be pumped with the fluid and placed into the fractures to support (prop open) said fractures. The proppant may be sourced from mines. Proppant is normally dried and sifted prior to being utilized in hydraulic fracturing operations to remove moisture and debris from the proppant. In some implementations, the hydraulic fracturing operations may utilize wet proppant systems. There has been an increase in the need for other types of proppant, such as wet proppant, proppant that has not been sifted, or any combination thereof.

### BRIEF DESCRIPTION OF THE DRAWINGS

Implementation of the disclosure may be better understood by referencing the accompanying drawings.

FIG. 1 is a block diagram illustrating a fracturing spread configured for hydraulically fracturing subsurface formations in one or more wells, according to some implementations.

FIGS. 2A-2B are illustrations of an example proppant transportation systems, according to some implementations.

FIG. 3 is a flowchart of example operations for determining characteristics of material being transported by a proppant transportation system and characteristics of the proppant transportation system, according to some implementations.

FIG. 4 is a block diagram of a YOLO-based proppant transportation monitoring system, according to some implementations.

FIG. 5 is a flowchart depicting example operations to configure a learning machine, according to some implementations.

FIG. 6 is a flowchart depicting example operations to train a learning machine, according to some implementations.

FIG. 7 is a block diagram depicting an example computer, according to some implementations.

### DESCRIPTION

The description that follows includes example systems, methods, techniques, and program flows that embody aspects of the disclosure. However, it is understood that this disclosure may be practiced without these specific details. For instance, this disclosure refers to one or more sensors positioned near a proppant transportation system to capture media content of the proppant transportation system for input into a you only look once (YOLO) object detection network to determine one or more material characteristics and proppant transportation system characteristics. Aspects of this disclosure can also be applied to any other suitable learning machine to determine properties of the fluid. For clarity, some well-known instruction instances, protocols, structures, and techniques have been omitted.

Example implementations relate to determining material characteristics and proppant transportation system characteristics during hydraulic fracturing operations. Proppant utilized in hydraulic fracturing operation may be sourced from sand mines or other sand manufacturing facilities. Proppant may include natural sand, man-made sand, or other man-made materials (such as ceramics). Prior to being utilized in hydraulic fracturing operations, the proppant may be dried and sifted to remove moisture and debris (such as rocks, sticks, metal, etc.) from the proppant. Wet proppant may cause a wide range of issues such as damage to the hydraulic fracturing equipment, reduced flow efficiency, etc. In some implementations, hydraulic fracturing operations may be designed to use wet proppant, and the equipment may need to be properly prepared for such operations. Debris present in the proppant may also cause issues as the proppant is transported through the hydraulic fracturing equipment such as equipment wear, unexpected failures, etc. In some implementations, in attempts to reduce costs, sand mines may have less processing such as a reduction in drying time and/or sifting of mined sand, resulting in wet sand and/or debris in sand used in the hydraulic fracturing operations. The ability to determine when operating with wet proppant and/or when debris is present in the proppant may assist in preventing damage to the hydraulic fracturing equipment, resulting in cost savings (i.e., from reduced repair and maintenance) and more efficient operations. In some implementations, it may also be beneficial to determine characteristics of the equipment moving and processing the proppant (i.e., the proppant transportation system such as the conveyor belt system) within the hydraulic fracturing operations. Due to the complex nature of the materials being transported and the proppant transportation system itself, approaches using conventional sensor-based systems may suffer in regard to accuracy of estimation and reliability. Moreover, the traditional sensor-based systems may be costly and, in some instances, not feasible.

In some implementations, a learning machine may be utilized to determine one or more characteristics of the material being transported by a proppant transportation system and characteristics of the proppant transportation system itself. During hydraulic fracturing operations, a proppant transportation system (such as a conveyor belt, screw, gravity feed, etc.) may transport proppant to a blender to be mixed with other materials such as water, chemicals, etc. before being pumped into the wellbore. One or more sensors may be positioned near the proppant transportation system to obtain media content (such as images, videos, etc.) of the material on or as it moves off of the proppant transportation system. Additionally, the sensors may obtain media content of the proppant transportation system itself. The learning machine may be configured to receive the media content as input, and trained to determine material characteristics (proppant characteristics and debris characteristics) and/or proppant transportation system characteristics as the proppant transportation system transports the material (proppant, debris (if present), etc.) to the blender. For example, the learning machine may identify if debris is present in the proppant, and determine the characteristics of said debris such as classification, size, shape, color, etc. or any combination thereof. Additionally, or alternatively, the material characteristics may include proppant characteristics such as proppant volume being transported by the proppant transportation system, proppant wetness, etc. The proppant transportation system characteristics may include characteristics of the belt, rollers, etc. indicating any wear or damage to the relative equipment.

In some implementations, the learning machine may be based on convolutional neural network (CNN) framework, such as networks similar to the you only look once (YOLO) object detection framework or any other suitable object detection frameworks. YOLO is utilized for its efficiency in real-time object detection. In some implementations, the learning machine may be configured with YOLO to identify and classify objects, material, etc. as they move along or out of the proppant transportation system. In some implementations, the spatial dimensions provided with YOLO bounding boxes of detected objects (such as debris, wet proppant, etc.) in combination with the perspective images of the proppant, debris (if detected), and proppant transportation system to estimate the volume and/or size of the detected objects. For example, the spatial dimensions provided by the YOLO bounding boxes in combination with the perspective of images may provide the volume of proppant being transported on a conveyor belt, the size of debris (such as the diameter of a rock) detected in the proppant, etc. In some implementations, the objects (detected by the YOLO) may be traced and timed such that the speed of the proppant transportation system (such as the conveyor belt) and/or the volume of proppant on the proppant transportation system may be estimated. For example, the speed of the conveyor belt may be determined. As another example, the tracing and timing of the proppant and/or debris may be utilized by the YOLO to determine the volume of the proppant on the conveyor belt (such as when belt slippage of the proppant may occur in a conveyor belt system, which may skew the volume of proppant and/or other material characteristic estimations when using traditional sensor systems).

In some implementations, the learning machine may be trained based on typical objects and their corresponding characteristics handled by the proppant transportation system. For example, training sample may include media content samples, with corresponding proppant characteristic samples, debris characteristic samples, and proppant transportation system characteristic samples. Proppant characteristic samples and debris characteristic samples may be labelled accordingly (i.e., wet proppant, dry proppant, with debris, without debris, etc.). In some implementations, the learning machine may be trained with training samples to determine textural changes and/or color changes in proppant and/or debris (when detected) which may be indicative of proppant wetness, types of debris, etc.

The learning machine may operate in real-time or in near real-time during hydraulic fracturing operations (i.e., while the proppant transportation system is in operation). Furthermore, the learning machine may be expanded to other characteristics and objects without requirement of additional sensors and/or extensive recalibration. For example, other characteristics of material transported along or out of the proppant transportation system, the proppant transportation system itself, etc. may be determined utilizing the sensors in place and the learning machine. In some implementations, in addition to the characteristics of the debris, proppant, and/or proppant transportation system, the learning machine may output an alarm for the corresponding characteristics. The alarm may be communicated to an operator to be analyzed and/or the alarm may be communicated to a system to which operations may be performed, altered, etc. In some implementations, the learning machine may output recommended actions based on the generated characteristics such as stopping operations, altering operations (e.g., controlling a conveyor belt speed, controlling material input/output, tub mixing, filtering and/or sifting, etc.). Similarly, the recommended actions may be communicated to an operator to be analyzed and/or the recommended actions may be communicated to a system to which operations may be implemented and/or activated.

In some implementations, a hydraulic fracturing operation or attribute on the surface may be modified or updated based on the material characteristics, proppant transportation system characteristics, and/or the corresponding alarms, recommended actions, etc. output from the learning machine. For example, an operation (at the surface or downhole) may be performed and/or directed to be performed to change a hydraulic fracturing operation or attribute based the material characteristics and/or proppant transportation system characteristics. For example, attributes of a hydraulic fracturing operation may be set based on whether the wetness of the proppant or debris is detected in the proppant. Examples of such attributes of the hydraulic fracturing operation may include belt proppant transportation system speed, material volume input/output onto/from the proppant transportation system, mixing in the blender tub, etc. For instance, if wet proppant is detected, any one of these attributes may be updated to allow for proper mixing of proppant with fluid and/or chemicals in the blender tub to generate the proper slurry mixture that is to be pumped into the wellbore.

While the aspects of the disclosure are described with reference to proppant transportation systems, it will be understood that these aspects are illustrative and that the scope of the claims is not limited to them. The learning machine described herein may be utilized for object detection in other material and systems within hydraulic fracturing operations. For example, one or more sensors may obtain media content of system configured to injected additives into the water (such as dry friction reducer), and detect, via the learning machine, characteristics of the additives such as moisture content and clumping.

### Example Systems

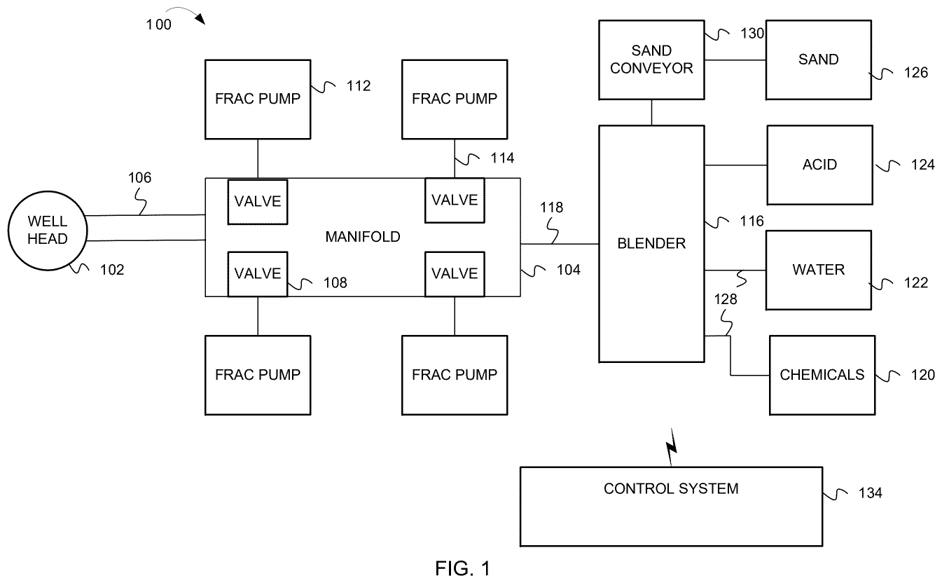

FIG. 1 is a block diagram illustrating a fracturing spread configured for hydraulically fracturing subsurface formations in one or more wells, according to some implementations. The fracturing spread described herein may be part of a larger system for drilling and fracturing well. The fracturing spread 100 may include a wellhead 102 that is connected to a wellbore. The wellbore (not shown) may be fluidically connected to one or more subsurface formations for the purpose of hydrocarbon recovery. Although FIG. 1 shows only one wellhead 102, there may be any suitable number of wellheads 102 and wellbores.

The wellhead 102 may be connected to a manifold 104 via piping 106. The piping 106 may include one or more pipes between the wellhead 102 and the manifold 104. The manifold 104 may include a plurality of valves 108 and various internal piping (not shown) for performing hydraulic fracturing operations.

The manifold 104 may be connected to one or more fracturing pumps (“frac pumps”) 112. The manifold 104 also may be connected to a blender 116 via piping 118. The blender 116 may be connected via piping 128 to one or more chemical containers 120, water containers 122, and acid containers 124. The blender 116 also may be connected to a proppant conveyor 130. In some implementations, one or more sensors may be positioned near the proppant conveyor 130 to obtain media content of the proppant and proppant conveyor 130 for input into a learning machine (as described below).

The fracturing spread 100 also may include a control system 134 configured to control one or more of the components of the fracturing spread 100. In some implementations, the control system 134 directly controls the equipment. However, the control system 134 may interact with various equipment controllers (not shown) and sensors to perform operations of the fracturing spread 100. For example, the fracturing spread 100 may include separate controllers (not shown) for the frac pumps 112, manifold 104, blender 116, and wellhead 102. The control system 134 may transmit commands to these separate controllers to change configurations (such as valve position, flow rate, chemical concentration, etc.) of the frac pumps 112, manifold 104, blender 116, chemical containers 120, etc.

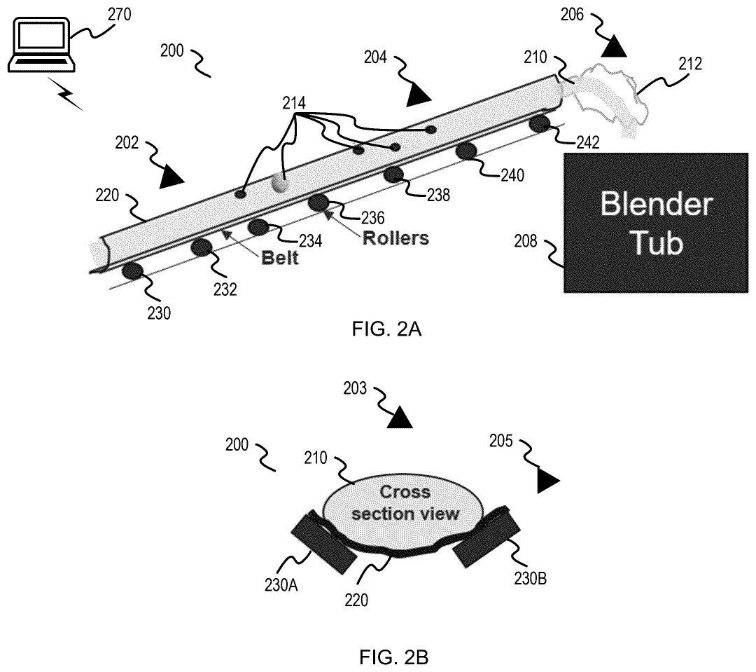

FIGS. 2A-2B are illustrations of an example proppant transportation systems, according to some implementations. In particular, FIG. 2A depicts a proppant transportation system 200. The proppant transportation system 200 depicted in FIGS. 2A-2B includes a conveyor belt system with a belt 220 and rollers 230-242 to transport material continuously and/or intermittently 210 (such as proppant and debris (if present)) to a blender tub 208 during hydraulic fracturing operations. The proppant transportation system 200 described herein is only one example configuration of a system capable of transporting material to the blender tub 208. Any other suitable transportation system configured for transporting materials may be used in hydraulic fracturing operations, such as a screw, proppant silo system (gravity feed system), etc.

The material 210 being transported on and into the blender tub 208 may include proppant (such as natural sand). In some implementations, the proppant may include debris 214 such as rocks, sticks, metal piece, nuts, bolts, etc. that was not filtered out prior to being placed onto the belt 220. In some implementations, the proppant may be wet or dry. For example, the proppant may not have been dried enough prior to being placed on the belt 220. The moisture level in the proppant may impact shape of the material 210 loaf and/or the dust 212 surrounding the material 210 loaf as the material comes off of the belt 220 and into the blender tub 208. For instance, the size of the dust 212 cloud may be inversely related to the moisture level in the proppant.

One or more sensors, such as sensors 202-206 may be positioned near the proppant transportation system 200. Each of the sensors 202-206 may be configured to obtain media content such as images, videos, etc. of the material 210 moving on the belt 220, the material 210 loaf coming off of the belt 220, the dust 212 surrounding the material 210 loaf, the belt 220 and rollers 230-242, and any other components within the proppant transportation system 200. In some implementations, the sensors may include cameras, RADAR sensors, light detection and ranging (LIDAR) sensors, etc. or any combination thereof to obtain the respective media content. Each of the sensors may obtain one or more pictures of the material 210 and/or the proppant transportation system 200 components at specified time intervals (i.e., 30 seconds, 5 minutes, 1 hour, etc.) for a period of time, record one or more videos (at any suitable frame rate) of the material 210, and/or the proppant transportation system 200 components for a period of time (such as whenever the proppant transportation system 200 is operating to transport material 210 to the blender tub 208), etc.

FIG. 2B depicts a cross-sectional view of the proppant transportation system 200. Rollers 230A and 230B may be cross-sectional views of the roller 230 depicted in FIG. 2A. In some implementations, the profile of the belt 220, rollers 230A, B, and/or material 210 in combination with the speed at which the belt 220 is moving may be indicative of the volume of material 210 on the belt 220. Sensors 203 and 205 may be configured to measure the dimensions (height, width) of the material 210 on the belt 220.

The sensors 202-206 may be communicatively coupled to a computer 270 configured to determine if debris 214 is present in the proppant, and determine characteristics of the material (debris, if present, and proppant) and the proppant transportation system (such as belt 220 and rollers 230-242). The computer 270 may be local or remote to the proppant transportation system 200. In some implementations, the processor of the computer 270 may be employed to run a learning machine for performing operations to determine if debris 214 is present in the proppant, and determine characteristics of the material (debris, if present, and proppant) and the proppant transportation system (such as belt 220 and rollers 230-242 based on the media content obtained by the sensors 202-206 (as further described below). The learning machine may be trained on media content of different proppant and debris samples to ensure its capability to recognize different debris, wet proppant, dry proppant, belt 220, roller condition, etc. In some implementations, the computer 270 may be configured to store the outputs from the learning machine in a database and/or to remote storage (i.e., the cloud). In some implementations, outputs from the learning machine may be displayed on a graphic user interface of the computer 270 or any remote device for further analysis. The computer 270 may be communicatively coupled to other components of the proppant transportation system to obtain data such as speed of the belt 220, roller rotation per minute (RPM), weight on the belt 220, etc.

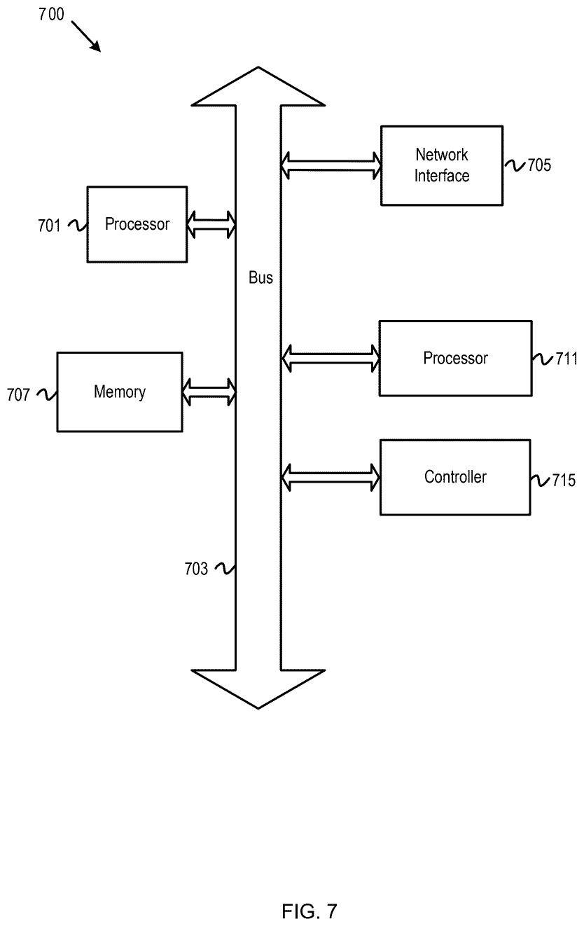

In some implementations, the processor of the computer 270 may control operations of the proppant transportation system 200 or subsequent operations. For instance, the processor of the computer 270 may control the belt 220, rollers 230-242, components for placing the material on the belt 20, etc. to control the volume of proppant added to the blender tub 208. The processor may then perform operations based on the material and proppant transportation system characteristics determined by the learning machine, such as adjusting the operations of the proppant transportation system 200, updating the maintenance schedule of one or more pumps in the hydraulic fracturing fleet. An example of the computer 270 is depicted in FIG. 7, which is further described below.

### Example Operations

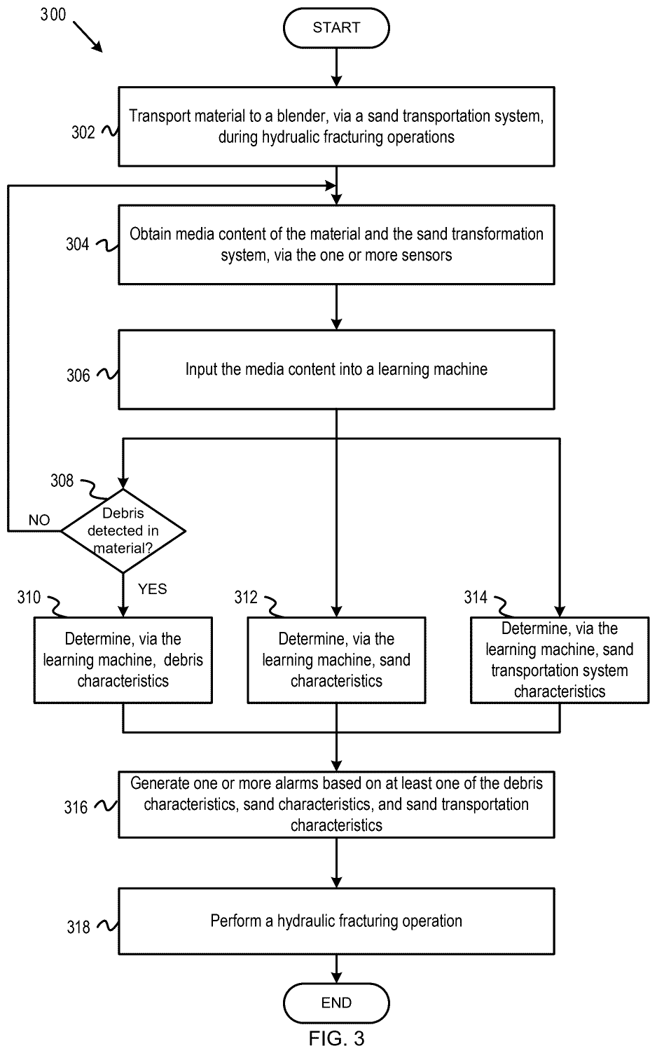

FIG. 3 is a flowchart of example operations for determining characteristics of material being transported by a proppant transportation system and characteristics of the proppant transportation system, according to some implementations. FIG. 3 depicts a flowchart 300 of operations to detect debris in proppant, debris characteristics, proppant characteristics, and proppant transportation characteristics, via a learning machine, during hydraulic fracturing operations. The operations of flowchart 300 are described in reference to the fracturing spread 100 of FIG. 1 and the computer 270 of FIG. 2. Additionally, the proppant transportation system described in the operations of the flowchart 300 is described in reference to the proppant transportation system 200 of FIGS. 2A-2B. Operations of the flowchart 300 begin at block 302.

At block 302, material may be transported to a blender, via a proppant transportation system, during hydraulic fracturing operations. Material, such as proppant, may be mixed in the blender with water, chemicals, etc. to create a slurry before being pumped into a wellbore. In some implementations, the material may include debris such as rocks, sticks, metal pieces, etc. that was not filtered out prior to being placed on the proppant transportation system. Additionally, or alternatively, the material may include moisture due to improper (or lack of) drying prior to being placed on the proppant transportation system. The proppant transportation system may include a conveyor belt system, proppant screw conveyor, gravity feed system, or any other system capable of transporting granular material.

At block 304, the processor of the computer 270 may obtain media content of the material and the proppant transformation system, via one or more sensors. The sensors may include cameras, RADAR, LIDAR, etc. or any combination thereof. The media content may include pictures, videos (such as MP4 or any suitable video format), etc. For example, the media content may include an MP4 video of the proppant transportation system captured in any suitable frame rate. The sensors may be positioned proximate the proppant transportation system and oriented such that media content of the material moving on the conveyor system, material moving off of the transportation system (and into the blender tub or the next piece of equipment in the fleet of hydraulic fracturing spread), components of the proppant transportation system (such as the belt, rollers, etc.), cross-sectional profiles of the material and/or proppant transportation system, etc. The media content may be obtained in time intervals (such as every second, 30 seconds, minute, etc.) such that media content of all material being transported may be captured. For example, pictures of the material and/or the proppant transportation system may be obtained at a time interval to ensure that no piece of debris may go undetected.

At block 306, the processor of the computer 270 may input the media content into a learning machine. In some implementations. The media content may be processed prior to being input into the learning machine. For example, if the media content includes videos, the video may be split into frames, and the frames may then be input into the learning machine. As described above, the learning machine may be any suitable object detection framework.

The learning machine may include a convolutional neural network (CNN). In some implementations, the learning machine may include a CNN with a network similar to you only look once (YOLO) object detection framework. YOLO is a real-time and/or near real-time object detection system that predicts bounding boxes and class probabilities directly from full images (or frames of a video) in a single evaluation. YOLO may be utilized to identify and classify objects (such as debris, sand dust, etc.), proppant characteristics (such as wet sand), proppant transformation system characteristics (such as belt wear) as the material moves along and off of the proppant transportation system. Spatial dimensions provided with YOLO bounding boxes of detected objects combined with the perspective of images/frames from the respective sensors to estimate debris and proppant characteristics such as size and volume, respectively. In some implementations, YOLO may trace the object and time to estimate the speed of the proppant transportation system transporting the material and/or the volume of the material on the proppant transportation system. In some implementations, the learning machine may reference speed sensors in the proppant transportation system to confirm and/or calibrate the speed of the conveyor system (belt, auger, etc.). Additional layers may be added to the network to capture other characteristics in the media content. For example, layers may be added to the network to recognize changes in texture, color, reflectiveness, etc. of objects which may be indicative of characteristics such as debris type, sand wetness, etc.

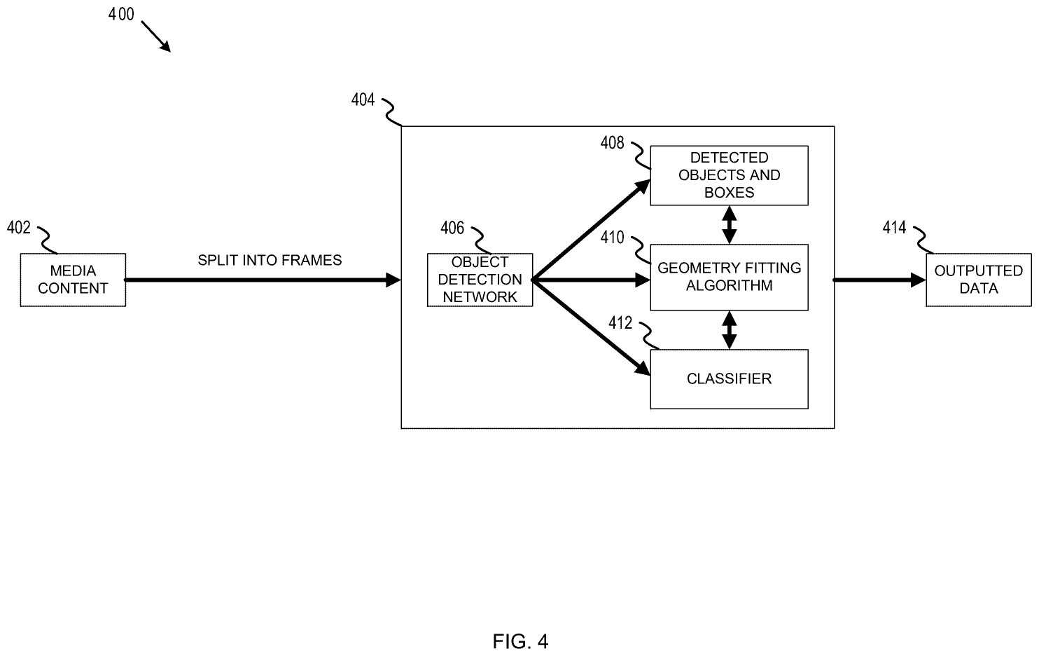

To help illustrate, FIG. 4 is a block diagram of a YOLO-based proppant transportation monitoring system, according to some implementations. In particular, FIG. 4 includes a YOLO-based system that includes a learning machine 404. Media content 402 is split into frames and input into the learning machine 404. The learning machine 404 includes an object detection network 406. The main module of the object detection network 406 may configured as a CNN. In some implementations, the CNN may comprise a YOLO module or any other suitable object detection framework to detect objects in the media content. Layers such as fully connected (FC) layers and convolutional (Conv) layers may be implemented into the object detection network 406 to integrate components such as multiclass classifier, bounding box regression, etc.

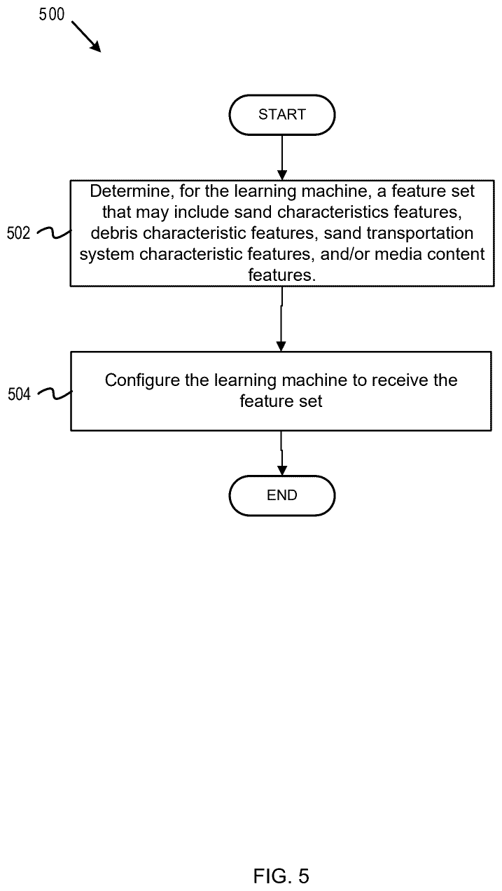

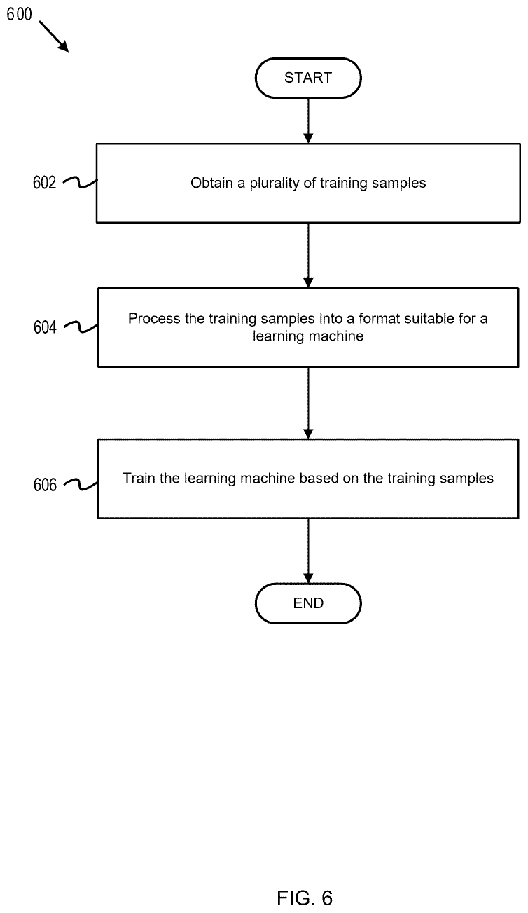

The object detection network 406 may also include, multiclass classifiers, bounding box classifiers, etc. or any combination thereof. The object detection network 406 may generate detected objects and boxes 408. For instance, the object detection network 406 may identify an object (such as a rock) in an image and generate a bounding box (via a bounding box regressor) around the object in the image. A geometry fitting algorithm 410 may be configured to identify the geometry of the object in the boxes from the detected objects and boxes 408 and subsequently determine the volume of the object. A classifier 412 may classify the object. In some implementations, a multiclass classifier may classify the object in an image and components of the learning machine may associate characteristics with identified object. For example, the objects detected in an image may be classified as a sand loaf and dust being disposed from a conveyor belt. Layers withing the CNN of the object detection network 406 may recognize the color and texture of the sand loaf and volume of the dust surrounding the sand loaf. Based on the objects and associated characteristics, the classifier 412 may classify the objects as wet sand and sand dust. The learning machine 404 may then generate the outputted data 414 comprising the classified objects and associated characteristics for an image. Configuration and training of the learning machine is described in FIGS. 5-6 below.

Operations of the flowchart 300 now proceed to blocks 308-314. The operations of block 308-314 may be performed in parallel or in series.

At block 308, the processor of the computer 270 may determine if debris is detecting in the material. For example, YOLO may divide an image, frame, etc. of the media content into a grid. If debris is present in one or more of the grid cells, the trained YOLO may then predict one or more bounding boxes for the object detected and generate a confidence score for each corresponding bounding box. The bounding box, confidence score, and any other suitable features may then be processed in the subsequent layers of the learning machine for classification. In some implementations, other features such as the perspective of the image, speed of the belt, etc. may be passed on to be utilized in determining the characteristics of the detected object. If debris is detected in the media content, then operations proceed to block 310. Otherwise, operations return to block 304.

At block 310, the processor of the computer 270 may determine, via the learning machine, debris characteristics. The learning machine may be trained to determine debris characteristics based on the training samples utilized to train the learning machine, as described in FIG. 6. Debris characteristics may include debris type, size, color, texture, etc. For example, YOLO may detect debris in the images of the material. The learning machine may classify the object and corresponding characteristics (such as a rock and the rock's size). As another example, the learning machine may determine the texture of an identified piece of debris is shiny with sharp edges. Accordingly, the learning machine may classify the debris as a piece of metal.

At block 312, the processor of the computer 270 may determine, via the learning machine, proppant characteristics. Similar to the debris detection, the learning machine may be trained to identify the proppant on the conveyor belt, sand loaf being thrown off of the conveyor belt, the sand dust surrounding the sand loaf, etc. The learning machine may then determine the characteristics of the objects based on the training samples utilized to train the learning machine, as described in FIG. 6. For example, YOLO may generate bounding boxes for detected objects relating to the proppant being transported on and off of the proppant transportation system. The learning machine may determine the proppant characteristics such as color, texture, pile size, dust cloud size, etc. Accordingly, the proppant characteristics may indicate the wetness of the proppant, volume of proppant on the conveyor belt, etc.

At block 314, the processor of the computer 270 may determine, via the learning machine, proppant transportation system characteristics. Similar to the operations in blocks 308-312, the learning machine may be trained to identify objects and associated characteristics of the proppant transportation system based on the training samples utilized to train the learning machine, as described in FIG. 6. For example, YOLO may generate bounding boxes for detected components in a proppant transportation system such as rollers, belt, etc. Accordingly, the learning machine may determine characteristics such as wear, deformation, defects, failures, etc. in the components of the proppant transportation system.

At block 316, the processor of the computer 270 may generate one or more alarms based on at least one of the debris characteristics, proppant characteristics, and proppant transportation system characteristics. The alarms may include different severity levels. For example, an alarm may include a warning, major, critical, extreme, etc. and/or have other levels indicating the seriousness such as green, yellow, red, etc. The alarms may correspond to characteristics such as type or size of debris, wetness of the proppant, wear on the belt, etc. For example, if one or more rocks above a specified diameter is detected, an appropriate level alarm may be generated to warn the operators that the rocks are present in the proppant which may result in damage to other equipment in the fleet such as plungers in the frac pumps.

At block 318, the processor of the computer 270 may perform a hydraulic fracturing operation. The hydraulic fracturing operation may be performed based on the debris, proppant, and/or proppant transportation system characteristics, the alarms, or any combination thereof. In some implementations, the hydraulic fracturing operations may be performed to mitigate any damage that may be caused to equipment in the fracturing fleet by the debris in the proppant, wet proppant, etc. For example, if debris is detected in the proppant, hydraulic fracturing attributes such as conveyor speed, proppant volume loaded onto the conveyor, etc. may be altered, stopped, etc. to prevent the debris from damaging equipment in the blender, pumps, etc. As another example, if the learning machine determines the belt and/or rollers are worn or deformed, operations may be modified to reduce the volume of proppant loaded onto the belt to prevent the belt and/or rollers from failing, or operations may be stopped to repair the damaged components. In some implementations, the pictures, frames, etc. of debris, proppant, and/or proppant transportation system in which objects have been detected and characterized by the learning machine may be displayed (i.e., on the graphic user interface of the computer 270 or any other suitable device, software, etc.) such that the objects may be analyzed. For example, pictures of objects characterized as rocks may be displayed for analysis and operations may be performed based on the analysis. Operations may be performed manually and/or automatically. For example, if wet proppant is detected, the computer 270 may communicate instructions to other systems to take action to account for the wet proppant, such as directing the proppant transportation system to adjust the speed of the conveyor, turning on one or more vibrators coupled with the system may to prevent proppant packing, or directing the blender to adjust water volumes to be mixed with the proppant to account for the moisture in the proppant. Alternatively, the operations may be manually performed. For example, an operator may stop the conveyor belt system to remove a rock from the proppant before it enters the blender tub. Any of the aforementioned operations may be performed automatically and/or manually.

In some implementations, the characteristics determined by the learning machine may be utilized to generate and/or update other models., For example, maintenance models may be updated based on the proppant characteristics. Proppant grain size, wetness, etc. may be mapped to frac pump performance (e.g., wear on the pump plungers after handling a slurry with type and wetness of proppant). Accordingly, a maintenance model of the pump may be adjusted when the identified proppant type, wetness, etc. is being used in hydraulic fracturing operations to prevent future failures, optimize the run time of the pump, and extend the life of the pump components.

In some implementations, the operations of the flowchart 300 may be repeated at different time intervals. For example, the operations of the flowchart 300 may be performed every second, 30 seconds, etc. to determine the characteristics mentioned above as proppant is transported through the surface equipment of hydraulic fracturing operations. In some implementations, the repetition of the operations of the flowchart 300 may correspond to the speed at which the proppant transportation system is moving. For example, the frequency of operations may increase as the speed of the conveyor belt increases to ensure media content of all material placed on the conveyor belt is obtained and processed (via the learning machine).

FIG. 5 is a flowchart depicting example operations to configure a learning machine, according to some implementations. FIG. 5 includes a flowchart 500 that may determine a feature set, and may configure the learning machine to receive the feature set as input. The learning machine may include any suitable learning machine such as a convolutional neural network (CNN) configured with a you only look once (YOLO) object detection framework. Operations of flowchart 500 of FIG. 5 are described in reference to the processor of the computer 270 of FIG. 2. Operations of the flowchart 500 start at block 502.

At block 502, the processor of the computer 270 may determine, for the learning machine, a feature set that may include proppant characteristics features, debris characteristic features, proppant transportation system characteristic features, and/or media content features. Proppant, debris, and proppant transportation system features may include features associated with the classification and characteristics of the aforementioned features. Proppant characteristic features may include color, texture, volume, sand loaf size, sand dust volume, grain size, etc. Debris features may include debris type, size, color, texture, etc. Proppant transportation system features may include belt profile, roller shape, roller wear, etc. A media content feature may include features associated with images, videos, etc. of the proppant transportation system and material being transported on and off of the proppant transportation system. Some implementations may utilize any suitable feature set including any suitable value related to the media content of the proppant transportation system and material being transported by the proppant transportation system.

At block 504, the processor of the computer 270 may configure the learning machine to receive the feature set as input. As noted, the features may include proppant characteristics features, debris characteristic features, proppant transportation system characteristic features, and/or media content features. The flowchart 500 ends after block 504.

After block 504, the learning machine may begin training itself based on training samples. The discussion of FIG. 6 provides additional details about training samples and training the learning machine.

FIG. 6 is a flowchart depicting example operations to train a learning machine, according to some implementations. FIG. 6 includes a flowchart 600 that may train a supervised and/or unsupervised learning machine with training samples. Operations of flowchart 600 of FIG. 6 are described in reference to the processor of the computer 270 of FIG. 2. Operations of the flowchart 600 start at block 602.

At block 602, the processor of the computer 270 may obtain a plurality of training samples. The training samples may ensure the learning machine's capability to recognize different objects and associated characteristics in material and the proppant transportation system. The training samples may include media content samples, proppant characteristic samples, debris characteristic samples, and proppant transportation system characteristic samples. The proppant characteristic samples, debris characteristic samples, proppant transportation system characteristic samples, and media content samples may be labeled with the object type and corresponding object characteristics (such as shape, color texture, volume, etc.). For example, an image of material on a conveyor belt may be obtained, where the material includes proppant and debris (such as sand and rocks). Object characteristics may be obtained. The proppant may be labeled as wet proppant, dry proppant, etc. and have characteristics such as color, texter, and grain size. Each piece of debris may be labeled as the debris type (such as a rock) and have characteristics such as diameter, color, and texture. The sample types and characteristics may be utilized as proppant characteristic samples and debris characteristic samples and the image of the material may be utilized as a media content sample for training the learning machine. The training samples may be generated by software and systems based on the system level design, numerical modeling, sample measurements, etc. For example, synthetic data may be generated and may be the labeled with proppant characteristics, debris characteristics, proppant transportation system characteristics, etc. to generate training samples. Training samples may be obtained from historical data such as media content of actual proppant transportation systems from historical hydraulic fracturing operations. Some implementations may utilize any suitable technique to obtain training samples.

At block 604, the processor of the computer 270 may process the training samples into a format suitable for a learning machine. For instance, if the learning machine is configured to accept inputs with values between 0 and 1, the fluid property sample may be scaled to values between 0 and 1.

At block 606, the processor of the computer 270 may train the learning machine based on the training samples. The learning machine may use fewer than all the training samples in its training process. For example, the learning machine may utilize 80% of the training samples at block 504. Later, the learning machine may use the remaining 20% of the training samples to test the learning machine. The learning machine may be updated (i.e., trained) as new training samples are obtained. For instance, the learning machine be trained with updated training samples obtained from synthetic data, historical testing data, etc.

While the aspects of the disclosure are described with reference to various implementations and exploitations, it will be understood that these aspects are illustrative and that the scope of the claims is not limited to them. In general, determining material and proppant transportation system characteristics via a learning machine as described herein may be implemented with facilities consistent with any hardware system or hardware systems. Many variations, modifications, additions, and improvements are possible.

Plural instances may be provided for components, operations or structures described herein as a single instance. Finally, boundaries between various components, operations and data stores are somewhat arbitrary, and particular operations are illustrated in the context of specific illustrative configurations. Other allocations of functionality are envisioned and may fall within the scope of the disclosure. In general, structures and functionality presented as separate components in the example configurations may be implemented as a combined structure or component. Similarly, structures and functionality presented as a single component may be implemented as separate components. These and other variations, modifications, additions, and improvements may fall within the scope of the disclosure.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example process in the form of a flow diagram. However, some operations may be omitted and/or other operations that are not depicted may be incorporated in the example processes that are schematically illustrated. For example, one or more additional operations may be performed before, after, simultaneously, or between any of the illustrated operations. In certain circumstances, multitasking and parallel processing may be advantageous. Moreover, the separation of various system components in the implementations described should not be understood as requiring such separation in all implementations, and the described program components and systems may generally be integrated together in a single software product or packaged into multiple software products. Additionally, other implementations are within the scope of the following claims. In some cases, the actions recited in the claims may be performed in a different order and still achieve desirable results.

### Example Computer

FIG. 7 is a block diagram depicting an example computer, according to some implementations. FIG. 7 depicts a computer 700 for determining proppant characteristics, debris characteristics, and proppant transportation characteristics of a proppant transportation system transporting debris during hydraulic fracturing operations. The computer 700 includes a processor 701 (possibly including multiple processors, multiple cores, multiple nodes, and/or implementing multi-threading, etc.). The computer 700 includes memory 707. The memory 707 may be system memory or any one or more of the above already described possible realizations of machine-readable media. The computer 700 also includes a bus 703 and a network interface 705. The computer 700 can communicate via transmissions to and/or from remote devices via the network interface 705 in accordance with a network protocol corresponding to the type of network interface, whether wired or wireless and depending upon the carrying medium. In addition, a communication or transmission can involve other layers of a communication protocol and or communication protocol suites (e.g., transmission control protocol, Internet Protocol, user datagram protocol, virtual private network protocols, etc.).

The computer 700 also includes a processor 711 and a controller 715 which may perform the operations described herein. For example, the processor 711 may obtain media content of material and a proppant transportation system and determine one or more characteristics of the material and the proppant transportation system. The controller 715 may perform an operation based on the characteristics of the material and the proppant transportation system, such as modifying hydraulic fracturing operations. The processor 711 and the controller 715 can be in communication. Any one of the previously described functionalities may be partially (or entirely) implemented in hardware and/or on the processor 701. For example, the functionality may be implemented with an application specific integrated circuit, in logic implemented in the processor 701, in a co-processor on a peripheral device or card, etc. Further, realizations may include fewer or additional components not illustrated in FIG. 7 (e.g., video cards, audio cards, additional network interfaces, peripheral devices, etc.). The processor 701 and the network interface 705 are coupled to the bus 703. Although illustrated as being coupled to the bus 703, the memory 707 may be coupled to the processor 701.

### Example Implementations

Implementation #1: A method to control hydraulic fracturing operations comprising: transporting material to a blender, via a proppant transportation system, during the hydraulic fracturing operations, wherein the material includes proppant; obtaining, via one or more sensors, media content of the material as the material is transported to the blender; and determining, via a learning machine, one or more material characteristics based on the media content.

Implementation #2: The method of Implementation #1, wherein the one or more material characteristics include one or more proppant characteristics and one or more debris characteristics, and wherein the proppant characteristics include at least one of proppant volume and proppant wetness.

Implementation #3: The method of Implementation #2 further comprising: detecting, via the learning machine, one or more debris present in the proppant; and determining, via the learning machine, the one or more debris characteristics based on the media content of the material, wherein the one or more debris characteristics include least one of debris size, debris shape, and debris color.

Implementation #4: The method of any one or more of Implementation #1-3 further comprising: obtaining, via the one or more sensors, the media content of the proppant transportation system; and determining, via the learning machine, proppant transportation system characteristics based on media content of the proppant transportation system, wherein the proppant transportation system characteristics include at least one of belt wear and roller wear.

Implementation #5: The method of any one or more of Implementation #1-4, wherein the media content of the material includes one or more pictures, videos, or any combination thereof from the respective sensors of the material being transported on the proppant transportation system and the material being transported off of the proppant transportation system.

Implementation #6: The method of any one or more of Implementation #1-5, wherein the proppant transportation system includes a conveyor system, a gravity feed system, or a screw system.

Implementation #7: The method of any one or more of Implementation #1-6 further comprising: determining, for the learning machine, a feature set including a proppant characteristics feature, a debris characteristic feature, a proppant transportation system characteristic feature, and a media feature; and configuring the learning machine to receive the feature set as input.

Implementation #8: The method of any one or more of Implementation #1-7 further comprising: training the learning machine to generate the one or more material characteristics based on a plurality of training samples, the training samples including media content samples, proppant characteristic samples, debris characteristic samples, and proppant transportation system characteristic samples.

Implementation #9: The method of any one or more of Implementation #1-8, wherein at least one of a hydraulic fracturing operation or a hydraulic fracturing attribute is modified based on the one or more material characteristics.

Implementation #10: A system comprising: a proppant transportation system configured to transport material to a blender during hydraulic fracturing operations; a processor; and a computer-readable medium having instructions stored thereon that are executable by the processor, the instructions including, instructions to obtain, via one or more sensors, media content of the material as the material is transported to the blender; and instructions to determine, via a learning machine, one or more material characteristics based on the media content.

Implementation #11: The system of Implementation #10, wherein the one or more material characteristics include one or more proppant characteristics and one or more debris characteristics, and wherein the proppant characteristics include at least one of proppant volume and proppant wetness.

Implementation #12: The system of Implementation #11 further comprising: instructions to detect, via the learning machine, one or more debris present in the material; and instructions to determine, via the learning machine, the one or more debris characteristics based on the media content of the material, wherein the one or more debris characteristics include least one of debris size, debris shape, and debris color.

Implementation #13: The system of c any one or more of Implementation #10-12 further comprising: instructions to obtain, via the one or more sensors, the media content of the proppant transportation system; instructions to determine, via the learning machine, proppant transportation system characteristics based on the media content of the proppant transportation system, wherein the proppant transportation system characteristics include at least one of belt wear and roller wear.

Implementation #14: The system of any one or more of Implementation #10-13, wherein the media content of the material includes one or more pictures, videos, or any combination thereof from the respective sensors of the material being transported on the proppant transportation system and the material being transported off of the proppant transportation system.

Implementation #15: The system of any one or more of Implementation #10-14, further comprising: instructions to direct an operation to modify at least one of the hydraulic fracturing operations or a hydraulic fracturing attribute based on the one or more material characteristics.

Implementation #16: A non-transitory, computer-readable medium having instructions stored thereon that are executable by a processor, the instructions comprising: instructions to transport material to a blender, via a proppant transportation system, during hydraulic fracturing operations, wherein the material includes proppant; instructions to obtain, via one or more sensors, media content of the material as the material is transported to the blender; and instructions to determine, via a learning machine, one or more material characteristics based on the media content.

Implementation #17: The non-transitory, computer-readable medium of Implementation #16, wherein the one or more material characteristics include one or more proppant characteristics and one or more debris characteristics, and wherein the proppant characteristics include at least one of proppant volume and proppant wetness.

Implementation #18: The non-transitory, computer-readable medium of Implementation #17 further comprising: instructions to detect, via the learning machine, one or more debris present in the proppant; and instructions to determine, via the learning machine, the one or more debris characteristics based on the media content of the material, wherein the one or more debris characteristics include least one of debris size, debris shape, and debris color.

Implementation #19: The non-transitory, computer-readable medium of any one or more of Implementation #16-18 further comprising: instructions to obtain, via the one or more sensors, the media content of the proppant transportation system; instructions to determine, via the learning machine, proppant transportation system characteristics based on the media content of the proppant transportation system, wherein the proppant transportation system characteristics include at least one of belt wear and roller wear.

Implementation #20: The non-transitory, computer-readable medium of any one or more of Implementation #16-19, further comprising: instructions to modify at least one of the hydraulic fracturing operations or a hydraulic fracturing attribute based on the one or more material characteristics.

Use of the phrase “at least one of” preceding a list with the conjunction “and” should not be treated as an exclusive list and should not be construed as a list of categories with one item from each category, unless specifically stated otherwise. A clause that recites “at least one of A, B, and C” can be infringed with only one of the listed items, multiple of the listed items, and one or more of the items in the list and another item not listed.

As used herein, the term “or” is inclusive unless otherwise explicitly noted. Thus, the phrase “at least one of A, B, or C” is satisfied by any element from the set {A, B, C} or any combination thereof, including multiples of any element.

1. A method to control hydraulic fracturing operations comprising:
transporting material to a blender, via a proppant transportation system, during the hydraulic fracturing operations, wherein the material includes proppant;
obtaining, via one or more sensors, media content of the material as the material is transported to the blender; and
determining, via a learning machine, one or more material characteristics based on the media content.

2. The method of claim 1, wherein the one or more material characteristics include one or more proppant characteristics and one or more debris characteristics, and wherein the proppant characteristics include at least one of proppant volume and proppant wetness.

3. The method of claim 2 further comprising:
detecting, via the learning machine, one or more debris present in the proppant; and
determining, via the learning machine, the one or more debris characteristics based on the media content of the material, wherein the one or more debris characteristics include least one of debris size, debris shape, and debris color.

4. The method of claim 1 further comprising:
obtaining, via the one or more sensors, the media content of the proppant transportation system; and
determining, via the learning machine, proppant transportation system characteristics based on media content of the proppant transportation system, wherein the proppant transportation system characteristics include at least one of belt wear and roller wear.

5. The method of claim 1, wherein the media content of the material includes one or more pictures, videos, or any combination thereof from the respective sensors of the material being transported on the proppant transportation system and the material being transported off of the proppant transportation system.

6. The method of claim 1, wherein the proppant transportation system includes a conveyor system, a gravity feed system, or a screw system.

7. The method of claim 1 further comprising:
determining, for the learning machine, a feature set including a proppant characteristics feature, a debris characteristic feature, a proppant transportation system characteristic feature, and a media feature; and
configuring the learning machine to receive the feature set as input.

8. The method of claim 1 further comprising:
training the learning machine to generate the one or more material characteristics based on a plurality of training samples, the training samples including media content samples, proppant characteristic samples, debris characteristic samples, and proppant transportation system characteristic samples.

9. The method of claim 1, wherein at least one of a hydraulic fracturing operation or a hydraulic fracturing attribute is modified based on the one or more material characteristics.

10. A system comprising:
a proppant transportation system configured to transport material to a blender during hydraulic fracturing operations;
a processor; and
a computer-readable medium having instructions stored thereon that are executable by the processor, the instructions including,
instructions to obtain, via one or more sensors, media content of the material as the material is transported to the blender; and
instructions to determine, via a learning machine, one or more material characteristics based on the media content.

11. The system of claim 10, wherein the one or more material characteristics include one or more proppant characteristics and one or more debris characteristics, and wherein the proppant characteristics include at least one of proppant volume and proppant wetness.

12. The system of claim 11 further comprising:
instructions to detect, via the learning machine, one or more debris present in the material; and
instructions to determine, via the learning machine, the one or more debris characteristics based on the media content of the material, wherein the one or more debris characteristics include least one of debris size, debris shape, and debris color.

13. The system of claim 10 further comprising:
instructions to obtain, via the one or more sensors, the media content of the proppant transportation system;
instructions to determine, via the learning machine, proppant transportation system characteristics based on the media content of the proppant transportation system, wherein the proppant transportation system characteristics include at least one of belt wear and roller wear.

14. The system of claim 10, wherein the media content of the material includes one or more pictures, videos, or any combination thereof from the respective sensors of the material being transported on the proppant transportation system and the material being transported off of the proppant transportation system.

15. The system of claim 10, further comprising:
instructions to direct an operation to modify at least one of the hydraulic fracturing operations or a hydraulic fracturing attribute based on the one or more material characteristics.

16. A non-transitory, computer-readable medium having instructions stored thereon that are executable by a processor, the instructions comprising:
instructions to transport material to a blender, via a proppant transportation system, during hydraulic fracturing operations, wherein the material includes proppant;
instructions to obtain, via one or more sensors, media content of the material as the material is transported to the blender; and
instructions to determine, via a learning machine, one or more material characteristics based on the media content.

17. The non-transitory, computer-readable medium of claim 16, wherein the one or more material characteristics include one or more proppant characteristics and one or more debris characteristics, and wherein the proppant characteristics include at least one of proppant volume and proppant wetness.

18. The non-transitory, computer-readable medium of claim 17 further comprising:
instructions to detect, via the learning machine, one or more debris present in the proppant; and
instructions to determine, via the learning machine, the one or more debris characteristics based on the media content of the material, wherein the one or more debris characteristics include least one of debris size, debris shape, and debris color.

19. The non-transitory, computer-readable medium of claim 16 further comprising:
instructions to obtain, via the one or more sensors, the media content of the proppant transportation system;
instructions to determine, via the learning machine, proppant transportation system characteristics based on the media content of the proppant transportation system, wherein the proppant transportation system characteristics include at least one of belt wear and roller wear.

20. The non-transitory, computer-readable medium of claim 16, further comprising:
instructions to modify at least one of the hydraulic fracturing operations or a hydraulic fracturing attribute based on the one or more material characteristics.
